(19) United States
(12) Patent Application Publication
(10) Pub. No.: US 2025/0022132 A1
(43) Pub. Date: Jan. 16, 2025

(54) SYSTEMS AND METHODS FOR IMAGE PROCESSING

(71) Applicant: SHANGHAI UNITED IMAGING HEALTHCARE CO., LTD., Shanghai (CN)

(72) Inventor: Chuang LUO, Shanghai (CN)

(73) Assignee: SHANGHAI UNITED IMAGING HEALTHCARE CO., LTD., Shanghai (CN)

(21) Appl. No.: 18/900,868

(22) Filed: Sep. 29, 2024

Related U.S. Application Data
(63) Continuation of application No. PCT/CN2023/097399, filed on May 31, 2023.

(30) Foreign Application Priority Data
May 31, 2022 (CN) 202210610623.5

(51) Int. Cl. G06T 7/00; G16H 30/40

(52) U.S. Cl. CPC G06T 7/0012; G16H 30/40; G06T 2200/24; G06T 2207/20092; G06T 2207/30004

(57) ABSTRACT

The present disclosure provides methods and systems for image processing. The methods may include determining a scanning protocol of a medical scan of a target subject. The scanning protocol may include a first parameter value of a first operation parameter of each of one or more post-processing operations. The first operation parameter of a post-processing operation may relate to whether the post-processing operation needs to be performed on target imaging data collected in the medical scan. The methods may include obtaining the target imaging data of the target subject collected in the medical scan that is performed according to the scanning protocol. The methods may further include performing at least part of the one or more post-processing operations on the target imaging data based on the first parameter value of the first operation parameter of each of the one or more post-processing operations.

### CROSS-REFERENCE TO RELATED APPLICATIONS

This application is a continuation of International Application No. PCT/CN2023/097399, filed on May 31, 2023, which claims priority to Chinese Patent Application No. 202210610623.5, filed on May 31, 2022, the contents of which are incorporated herein by reference.

### TECHNICAL FIELD

The present disclosure generally relates to image processing, and more particularly, relates to systems and methods for performing post-processing operations on imaging data.

### BACKGROUND

Medical imaging techniques (e.g., a computed tomography (CT) technique, a positron emission tomography (PET) technique, a magnetic resonance (MR) technique, etc.) may be used to provide detection information (e.g., anatomical information, functional information, etc.) of a target subject. For example, after imaging data of the target subject is acquired using the medical imaging techniques, a doctor may obtain an analysis report including the detection information by performing one or more post-processing operations on the imaging data. However, the one or more post-processing operations may be performed on the acquired imaging data. The reliability of the acquisition of the imaging data and/or image quality of the imaging data cannot be ensured, which reduces the accuracy of the detection information determination. In addition, the doctor needs input instructions to determine which post-processing operation(s) need to be performed, and the type of data based on which each post-processing operation is performed, which is time-consuming, labor-intensive, and inefficient.

Therefore, it is desirable to provide systems and methods for image processing, thereby improving the accuracy of the detection information determination.

### SUMMARY

In an aspect of the present disclosure, a method for image processing is provided. The method may be implemented on a computing device having at least one processor and at least one storage device. The method may include determining a scanning protocol of a medical scan of a target subject. The scanning protocol may include a first parameter value of a first operation parameter of each of one or more post-processing operations. The first operation parameter of a post-processing operation may relate to whether the post-processing operation needs to be performed on target imaging data collected in the medical scan. The method may include obtaining the target imaging data of the target subject collected in the medical scan that is performed according to the scanning protocol. The method may further include performing at least part of the one or more post-processing operations on the target imaging data based on the first parameter value of the first operation parameter of each of the one or more post-processing operations.

In some embodiments, the one or more post-processing operations may include at least one of an organizational analysis, a region of interest (ROI) segmentation, a noise reduction, or a resolution adjustment.

In some embodiments, the scanning protocol may further include a parameter value of a system parameter corresponding to the target subject. The system parameter may relate to whether imaging data relating to the target subject needs to be post-processed. The performing at least part of the one or more post-processing operations on the target imaging data may include determining whether the target imaging data needs to be post-processed based on the parameter value of the system parameter; in response to determining that the target imaging data needs to be post-processed, performing the at least part of the one or more post-processing operations on the target imaging data based on the first parameter value of the first operation parameter of each of the one or more post-processing operations.

In some embodiments, the first parameter value of the first operation parameter of each of the one or more post-processing operations may be determined by: for each of the one or more post-processing operations, determining a first recommendation value of the first operation parameter of the post-processing operation, and determining the first parameter value of the first operation parameter of the post-processing operation based on the first recommendation value and a user input.

In some embodiments, the determining a first recommendation value of the first operation parameter of the post-processing operation may include determining the first recommendation value of the first operation parameter of the post-processing operation based on feature information relating to the target subject.

In some embodiments, the performing at least part of the one or more post-processing operations on the target imaging data based on the first parameter value of the first operation parameter of each of the one or more post-processing operations may include generating an analysis result by performing abnormity analysis on the target imaging data; determining a second parameter value of the first operation parameter of each of the one or more post-processing operations based on the first parameter value of the first operation parameter of each of the one or more post-processing operations and the analysis result; and performing the at least part of the one or more post-processing operations on the target imaging data based on the second parameter value of the first operation parameter of each of the one or more post-processing operations.

In some embodiments, the generating an analysis result by performing an abnormity analysis on the target imaging data may include obtaining an abnormity analysis model, the abnormity analysis model being a trained machine learning model; and generating the analysis result based on the target imaging data using the abnormity analysis model.

In some embodiments, the scanning protocol may include a third parameter value of a second operation parameter of each of the one or more post-processing operations. The second operation parameter of a post-processing operation may relate to the type of data based on which the post-processing operation is performed.

In some embodiments, the third parameter value of the second operation parameter of each of the one or more post-processing operations may be determined by: for each of the one or more post-processing operations, determining a second recommendation value of the second operation parameter of the post-processing operation, and determining the third parameter value of the post-processing operation based on the second recommendation value and a user input.

In some embodiments, the method may further include generating an analysis report of the target subject based on post-processed imaging data.

In some embodiments, the method may further include displaying the scanning protocol of the medical scan through a user interface.

In another aspect of the present disclosure, a system for image processing is provided. The system may include at least one storage device including a set of instructions; and at least one processor configured to communicate with the at least one storage device. When executing the set of instructions, the at least one processor may be configured to direct the system to perform operations. The operations may include determining a scanning protocol of a medical scan of a target subject. The scanning protocol may include a first parameter value of a first operation parameter of each of one or more post-processing operations. The first operation parameter of a post-processing operation may relate to whether the post-processing operation needs to be performed on target imaging data collected in the medical scan. The operations may include obtaining the target imaging data of the target subject collected in the medical scan that is performed according to the scanning protocol. The operations may further include performing at least part of the one or more post-processing operations on the target imaging data based on the first parameter value of the first operation parameter of each of the one or more post-processing operations.

In still another aspect of the present disclosure, a system for image processing is provided. The system may include a determination module, an obtaining module, and a post-processing module. The determination module may be configured to determine a scanning protocol of a medical scan of a target subject. The scanning protocol may include a first parameter value of a first operation parameter of each of one or more post-processing operations. The first operation parameter of a post-processing operation may relate to whether the post-processing operation needs to be performed on target imaging data collected in the medical scan. The obtaining module may be configured to obtain the target imaging data of the target subject collected in the medical scan that is performed according to the scanning protocol. The post-processing module may be configured to perform at least part of the one or more post-processing operations on the target imaging data based on the first parameter value of the first operation parameter of each of the one or more post-processing operations.

In still another aspect of the present disclosure, a non-transitory computer readable medium is provided. The non-transitory computer readable medium may include executable instructions that, when executed by at least one processor, direct the at least one processor to perform a method. The method may include determining a scanning protocol of a medical scan of a target subject. The scanning protocol may include a first parameter value of a first operation parameter of each of one or more post-processing operations. The first operation parameter of a post-processing operation may relate to whether the post-processing operation needs to be performed on target imaging data collected in the medical scan. The method may include obtaining the target imaging data of the target subject collected in the medical scan that is performed according to the scanning protocol. The method may further include performing at least part of the one or more post-processing operations on the target imaging data based on the first parameter value of the first operation parameter of each of the one or more post-processing operations.

In still another aspect of the present disclosure, a device for image processing is provided. The system may include at least one storage device including a set of instructions; and at least one processor configured to communicate with the at least one storage device. When executing the set of instructions, the at least one processor may be configured to direct the device to perform operations. The operations may include determining a scanning protocol of a medical scan of a target subject. The scanning protocol may include a first parameter value of a first operation parameter of each of one or more post-processing operations. The first operation parameter of a post-processing operation may relate to whether the post-processing operation needs to be performed on target imaging data collected in the medical scan. The operations may include obtaining the target imaging data of the target subject collected in the medical scan that is performed according to the scanning protocol. The operations may further include performing at least part of the one or more post-processing operations on the target imaging data based on the first parameter value of the first operation parameter of each of the one or more post-processing operations.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary imaging system according to some embodiments of the present disclosure;

FIG. 2 is a block diagram illustrating an exemplary processing device according to some embodiments of the present disclosure;

FIG. 3 is a flowchart illustrating an exemplary process for image processing according to some embodiments of the present disclosure;

FIG. 4 is a schematic diagram illustrating an exemplary user interface according to some embodiments of the present disclosure;

FIG. 5 is a flowchart illustrating an exemplary process for determining a first parameter value of a first operation parameter according to some embodiments of the present disclosure;

FIG. 6 is a flowchart illustrating an exemplary process for image processing according to some embodiments of the present disclosure;

FIG. 7 is a schematic diagram illustrating an exemplary process for determining a parameter value of a first operation parameter according to some embodiments of the present disclosure;

FIG. 8 is a flowchart illustrating an exemplary process for determining a third parameter value of a second operation parameter according to some embodiments of the present disclosure; and

FIG. 9 is a schematic diagram illustrating an exemplary user interface for ROI segmentation according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

These and other features, and characteristics of the present disclosure, as well as the methods of operation and functions of the related elements of structure and the combination of parts and economics of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

The present disclosure relates to systems and methods for image processing. The methods may include determining a scanning protocol of a medical scan of a target subject. The scanning protocol may include a first parameter value of a first operation parameter of each of one or more post-processing operations. The first operation parameter of a post-processing operation may relate to whether the post-processing operation needs to be performed on target imaging data collected in the medical scan. The methods may include obtaining the target imaging data of the target subject collected in the medical scan that is performed according to the scanning protocol. The methods may further include performing at least part of the one or more post-processing operations on the target imaging data based on the first parameter value of the first operation parameter of each of the one or more post-processing operations. In some embodiments, the scanning protocol may further include a third parameter value of a second operation parameter of each of the one or more post-processing operations. The second operation parameter of a post-processing operation may relate to the type of data based on which the post-processing operation is performed.

According to some embodiments of the present disclosure, by determining the first parameter value and the third parameter value of each of the one or more post-processing operations, post-processing operations that need to be performed and what type of data each post-processing operation is performed based on may be determined, which improves the reliability of the post-processing operations and subsequent image analysis. In addition, the first parameter value and the third parameter value may be determined before the target imaging data is obtained, and no instruction needs to be input by a user after the target imaging data is obtained, which reduces the labor consumption and simplifies the process of the post-processing operations, thereby improving the efficiency of the image processing.

In some embodiments, the first parameter value and the third parameter value may be determined automatically. Alternatively, the first parameter value and the third parameter value may be determined according to a configuration instruction and/or a user instruction corresponding to specific conditions (e.g., a type of symptom, a type of patient, etc.), which improve the efficiency of parameter value determination, thereby improving the accuracy and efficiency of the image analysis.

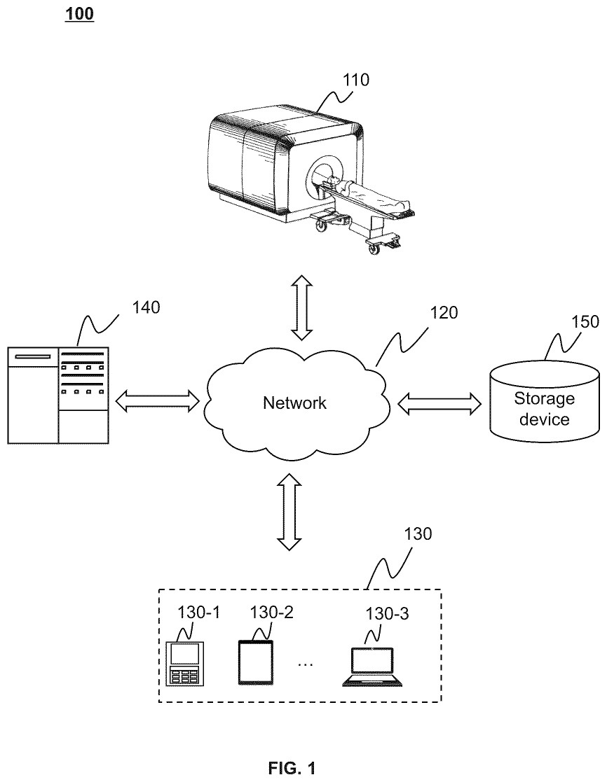

FIG. 1 is a schematic diagram illustrating an exemplary imaging system 100 according to some embodiments of the present disclosure. As shown in FIG. 1, the imaging system 100 may include an imaging device 110, a network 120, one or more terminals 130, a processing device 140, and a storage device 150. In some embodiments, the imaging device 110, the processing device 140, the storage device 150, and/or the terminal(s) 130 may be connected to and/or communicate with each other via a wireless connection (e.g., the network 120), a wired connection, or a combination thereof. The connection between the components in the imaging system 100 may be variable.

The imaging device 110 may be configured to generate or provide imaging data by scanning a target subject or at least a part of the target subject. For example, the imaging device 110 may collect imaging data of a target subject in a medical scan that is performed according to a scanning protocol of the medical scan of the target subject. In some embodiments, the imaging device 110 may include a single modality imaging device. For example, the imaging device 110 may include a positron emission tomography (PET) device, a single-photon emission computed tomography (SPECT) device, a computed tomography (CT) device, a magnetic resonance (MR) device, a b-scan ultrasonography device, a thermal texture maps (TTM) device, a medical electronic endoscope (MEE) device, or the like. In some embodiments, the imaging device 110 may include a multi-modality imaging device. Exemplary multi-modality imaging devices may include a positron emission tomography-computed tomography (PET-CT) device, a positron emission tomography-magnetic resonance imaging (PET-MRI) device, a single-photon emission computed tomography-computed tomography (SPECT-CT) device, etc. The multi-modality scanner may perform multi-modality imaging simultaneously or in sequence. For example, the PET-CT device may generate structural X-ray CT image data and functional PET image data simultaneously or in sequence. The PET-MRI device may generate MRI data and PET data simultaneously or in sequence.

The target subject may include patients or other experimental subjects (e.g., experimental mice or other animals). In some embodiments, the target subject may be a patient or a specific portion, organ, and/or tissue of the patient. For example, the target subject may include the head, the neck, the thorax, the heart, the stomach, a blood vessel, soft tissue, a tumor, nodules, or the like, or any combination thereof. In some embodiments, the target subject may be non-biological. For example, the target subject may include a phantom, a man-made object, etc.

The network 120 may include any suitable network that can facilitate the exchange of information and/or data for the imaging system 100. In some embodiments, one or more components (e.g., the imaging device 110, the terminal 130, the processing device 140, the storage device 150, etc.) of the imaging system 100 may communicate information and/or data with one or more other components of the imaging system 100 via the network 120. For example, the processing device 140 may obtain image data from the imaging device 110 via the network 120. As another example, the processing device 140 may obtain user instructions from the terminal 130 via the network 120. In some embodiments, the network 120 may include one or more network access points.

The terminal(s) 130 may include a mobile device 130-1, a tablet computer 130-2, a laptop computer 130-3, or the like, or any combination thereof. In some embodiments, the mobile device 130-1 may include a smart home device, a wearable device, a mobile device, a virtual reality device, an augmented reality device, or the like, or any combination thereof. In some embodiments, the terminal(s) 130 may include a processing unit, a display unit, an input/output (I/O) unit, a storage unit, etc. Exemplary display units may include a liquid crystal display (LCD), a light-emitting diode (LED)-based display, a flat panel display, a curved screen, a television device, a cathode ray tube (CRT), or the like, or a combination thereof. In some embodiments, the display unit may include an interactive interface that is configured to receive an input (e.g., a user instruction) from a user. In some embodiments, the terminal(s) 130 may be part of the processing device 140.

The processing device 140 may process data and/or information obtained from one or more components (the imaging device 110, the terminal(s) 130, and/or the storage device 150) of the imaging system 100. For example, the processing device 140 may perform one or more post-processing operations on target imaging data of a target subject based on a scanning protocol of a medical scan of the target subject. The one or more post-processing operations may include an organizational analysis, a region of interest (ROI) segmentation, a noise reduction, a resolution adjustment, or the like, or any combination thereof. As another example, the processing device 140 may generate an analysis report of the target subject based on post-processed imaging data. In some embodiments, the processing device 140 may be a single server or a server group. The server group may be centralized or distributed. In some embodiments, the processing device 140 may be local or remote. In some embodiments, the processing device 140 may be implemented on a cloud platform.

In some embodiments, the processing device 140 may be implemented by a computing device. For example, the computing device may include a processor, a storage, an input/output (I/O), and a communication port. The processor may execute computer instructions (e.g., program codes) and perform functions of the processing device 140 in accordance with the techniques described herein. The computer instructions may include, for example, routines, programs, objects, components, data structures, procedures, modules, and functions, which perform particular functions described herein. In some embodiments, the processing device 140, or a portion of the processing device 140 may be implemented by a portion of the terminal 130.

The storage device 150 may store data/information obtained from the imaging device 110, the terminal(s) 130, and/or any other component of the imaging system 100. In some embodiments, the storage device 150 may include a mass storage, a removable storage, a volatile read-and-write memory, a read-only memory (ROM), or the like, or any combination thereof. In some embodiments, the storage device 150 may store one or more programs and/or instructions to perform exemplary methods described in the present disclosure.

In some embodiments, the imaging system 100 may include one or more additional components and/or one or more components of the imaging system 100 described above may be omitted. Additionally or alternatively, two or more components of the imaging system 100 may be integrated into a single component. A component of the imaging system 100 may be implemented on two or more sub-components.

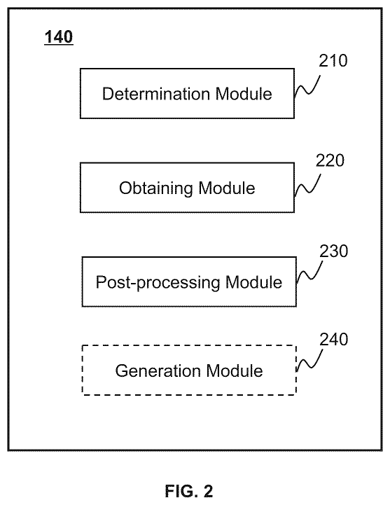

FIG. 2 is a block diagram illustrating an exemplary processing device 140 according to some embodiments of the present disclosure. The modules illustrated in FIG. 2 may be implemented on the processing device 140. In some embodiments, the processing device 140 may be in communication with a computer-readable storage medium (e.g., the storage device 150 illustrated in FIG. 1) and execute instructions stored in the computer-readable storage medium. The processing device 140 may include a determination module 210, an obtaining module 220, and a post-processing module 230.

The determination module 210 may be configured to determine a scanning protocol of a medical scan of a target subject. The scanning protocol may be used to guide the implementation of the medical scan of the target subject and/or the processing of target imaging data collected in the medical scan. For example, the scanning protocol may include one or more parameters to be used in the medical scan of the target subject and/or the process of the target imaging data. Exemplary parameters may include an operation parameter, a system parameter corresponding to the target subject, a scanning parameter, or the like, or any combination thereof. More descriptions regarding the determination of the scanning protocol of the medical scan of the target subject may be found elsewhere in the present disclosure. Sec, e.g., operation 302 and relevant descriptions thereof.

The obtaining module 220 may be configured to obtain the target imaging data of the target subject collected in the medical scan that is performed according to the scanning protocol. The target imaging data may refer to imaging data that needs to be analyzed and/or post-processed. More descriptions regarding the obtaining the target imaging data of the target subject may be found elsewhere in the present disclosure. See, e.g., operation 304 and relevant descriptions thereof.

The post-processing module 230 may be configured to perform at least part of the one or more post-processing operations on the target imaging data based on the scanning protocol. More descriptions regarding the performing the at least part of the one or more post-processing operations may be found elsewhere in the present disclosure. Sec, e.g., operation 306 and relevant descriptions thereof.

In some embodiments, the processing device 140 may further include a generation module 240. The generation module 240 may be configured to generate an analysis report of the target subject based on post-processed imaging data. The analysis report may include an organizational analysis result, an ROI segmentation result, detection information, or the like, or any combination thereof. More descriptions regarding the generation of the analysis report of the target subject may be found elsewhere in the present disclosure. See, e.g., operation 308 and relevant descriptions thereof.

It should be noted that the above descriptions of the processing device 140 are provided for the purposes of illustration, and are not intended to limit the scope of the present disclosure. For persons having ordinary skills in the art, various variations and modifications may be conducted under the guidance of the present disclosure. However, those variations and modifications do not depart from the scope of the present disclosure. In some embodiments, the processing device 140 may include one or more other modules. For example, the processing device 140 may include a storage module to store data generated by the modules in the processing device 140. In some embodiments, any two of the modules may be combined as a single module, and any one of the modules may be divided into two or more units.

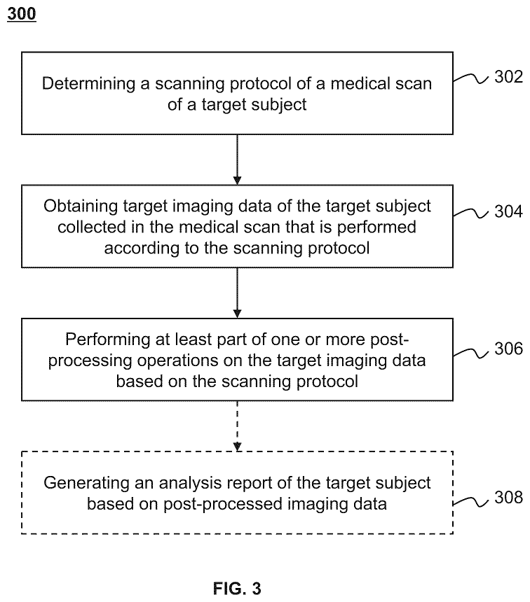

FIG. 3 is a flowchart illustrating an exemplary process for image processing according to some embodiments of the present disclosure. Process 300 may be implemented in the imaging system 100 illustrated in FIG. 1. For example, the process 300 may be stored in the storage device 150 in the form of instructions (e.g., an application), and invoked and/or executed by the processing device 140.

Medical imaging techniques may be used to provide detection information of a target subject. For example, after imaging data of the target subject is acquired using the medical imaging techniques, one or more post-processing operations (e.g., an organizational analysis) may be performed on the imaging data to obtain the detection information of the target subject. However, the one or more post-processing operations may be performed on the acquired imaging data. The reliability of the acquisition of the imaging data and/or image quality of the imaging data cannot be ensured, which reduces the accuracy of the detection information determination. In addition, parameters relating to the one or more post-processing operations are normally predefined according to the system default setting and cannot be adjusted, which further reduces the accuracy of the image processing and/or the detection information determination. In order to improve the accuracy and efficiency of the detection information determination, the process 300 may be performed.

In 302, the processing device 140 (e.g., the determination module 210) may determine a scanning protocol of a medical scan of a target subject.

The scanning protocol may be used to guide the implementation of the medical scan of the target subject and/or the processing of target imaging data collected in the medical scan. For example, the scanning protocol may include one or more parameters to be used in the medical scan of the target subject and/or the process of the target imaging data. Exemplary parameters may include an operation parameter, a system parameter corresponding to the target subject, a scanning parameter, or the like, or any combination thereof.

The operation parameter may include one or more parameters relating to one or more post-processing operations. For example, the operation parameter may include a first operation parameter of each of the one or more post-processing operations, a second operation parameter of each of the one or more post-processing operations, etc.

The one or more post-processing operations may include an organizational analysis, a region of interest (ROI) segmentation (or an ROI identification), a noise reduction, a resolution adjustment, or the like, or any combination thereof. The organizational analysis may refer to an operation that analyzes organizational content(s) and/or organizational distribution(s) of the target subject or a portion (e.g., an ROI) of the target subject. Exemplary organizational analyses may include an analysis of fat tissue, an analysis of muscle tissue, an analysis of nerve tissue, an analysis of epithelial tissue, an analysis of connective tissue, or the like, or any combination thereof. The ROI segmentation may refer to an operation that segments (or identifies) one or more ROIs from the target imaging data. The noise reduction may refer to an operation that reduces the noise in the target imaging data. The resolution adjustment may refer to an operation that adjusts (e.g., increases or decreases) the image resolution of the target imaging data.

The first operation parameter of a post-processing operation may relate to whether the post-processing operation needs to be performed on the target imaging data. For example, when the post-processing operation is the organizational analysis, the first operation parameter of the organizational analysis may relate to whether the organizational analysis needs to be performed on the target imaging data. As another example, when the post-processing operation is the ROI segmentation, the first operation parameter of the organizational analysis may relate to whether the ROI segmentation needs to be performed on the target imaging data.

In some embodiments, the scanning protocol may include a first parameter value of the first operation parameter of each of the one or more post-processing operations. The first parameter value may indicate whether the post-processing operation needs to be performed on the target imaging data. For example, the first parameter value may include a parameter value indicating that the post-processing operation needs to be performed on the target imaging data, and a parameter value indicating that the post-processing operation does not need to be performed on the target imaging data. Merely by way of example, when the post-processing operation is the organizational analysis, the first parameter value of the first operation parameter of the organizational analysis may include a parameter value indicating that the organizational analysis needs to be performed on the target imaging data, and a parameter value indicating that the organizational analysis does not need to be performed on the target imaging data.

In some embodiments, the first parameter value may be represented as numbers, letters, symbols, etc. For example, the first parameter value of 1 may indicate that the post-processing operation needs to be performed on the target imaging data, and the first parameter value of 0 may indicate that the post-processing operation does not need to be performed on the target imaging data.

In some embodiments, the first parameter value of the first operation parameter of each of the one or more post-processing operations may be determined based on a system default setting or set manually by the user. For example, for each of the one or more post-processing operations, the processing device 140 may determine a first recommendation value of the first operation parameter of the post-processing operation, and determine the first parameter value of the first operation parameter of the post-processing operation based on the first recommendation value and a user input. More descriptions regarding the determination of the first parameter value of the first operation parameter may be found in elsewhere in the present disclosure (e.g., FIG. 5 and the descriptions thereof).

The second operation parameter of a post-processing operation may relate to the type of data based on which the post-processing operation is performed. The type of data based on which the post-processing operation is performed may include the target imaging data or post-processed imaging data generated by performing one or more other post-processing operations on the target imaging data.

In some embodiments, the scanning protocol may include a third parameter value of the second operation parameter of each of the one or more post-processing operations. The third parameter value may indicate the type of data based on which the post-processing operation is performed. For example, when the post-processing operation is the organizational analysis, the third parameter value of the second operation parameter of the organizational analysis may be the target imaging data or post-processed imaging data generated by performing at least one of the ROI segmentation, the noise reduction, and the resolution adjustment on the target imaging data. As another example, when the post-processing operation is the ROI segmentation, the third parameter value of the second operation parameter of the organizational analysis may be the target imaging data or post-processed imaging data generated by performing at least one of the noise reduction and the resolution adjustment on the target imaging data.

In some embodiments, the third parameter value of a post-processing operation may be represented as numbers, letters, symbols, etc. For example, the third parameter value of 0 may indicate that the post-processing operation needs to be performed directly on the target imaging data, the third parameter value of 1 may indicate that the post-processing operation needs to be performed on segmented imaging data generated by performing the ROI segmentation on the target imaging data, and the third parameter value of 2 may indicate that the post-processing operation needs to be performed based on noise reduction imaging data generated by performing the noise reduction on the target imaging data. For example, assuming that the post-processing operation is the resolution adjustment, when the third parameter value of the second operation parameter of the resolution adjustment is 1, the ROI segmentation may be firstly performed on the target imaging data to generate segmented imaging data, and then the resolution adjustment may be performed on the segmented imaging data.

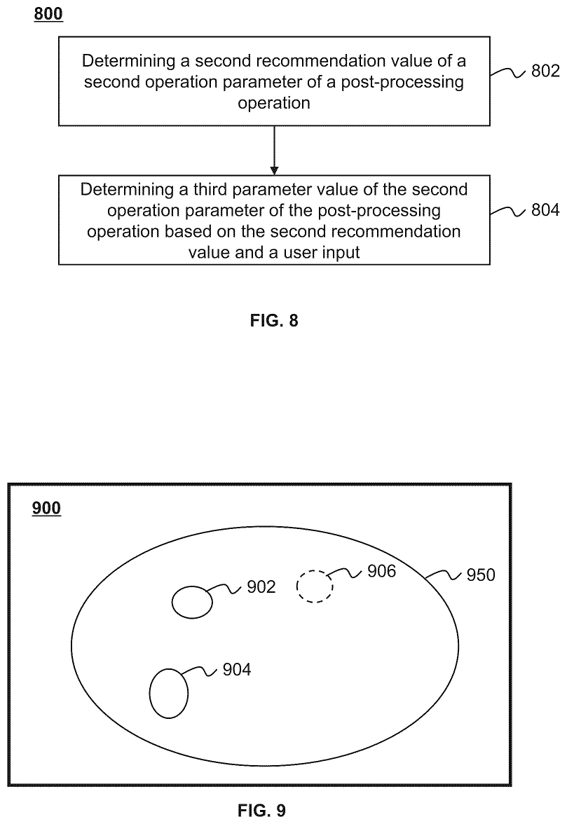

In some embodiments, the third parameter value of the second operation parameter of each of the one or more post-processing operations may be determined based on a system default setting or set manually by the user. For example, for each of the one or more post-processing operations, the processing device 140 may determine a second recommendation value of the second operation parameter of the post-processing operation, and determine the third parameter value of the second operation parameter of the post-processing operation based on the second recommendation value and a user input. More descriptions regarding the determination of the third parameter value of the second operation parameter may be found in elsewhere in the present disclosure (e.g., FIG. 8 and the descriptions thereof).

The system parameter may relate to whether imaging data relating to the target subject (e.g., the target imaging data to be collected in the medical scan and other imaging data collected in other scans) needs to be post-processed. In some embodiments, the system parameter may be a customized parameter set for a same target subject. That is, when the target subject is determined, a parameter value of the system parameter may be determined. Since a plurality of sets of imaging data corresponding to one target subject have similar or same scanning features, the processing device 140 may determine the parameter value of the system parameter of the target subject so that different sets of imaging data of the target subject collected in different scans can be processed according to the parameter value. By using the system parameter corresponding to the target subject instead of using a system parameter corresponding to each medical scan, there is no need to redetermine the system parameter each time a new set of imaging data of the target subject is collected, which improves the convenience and efficiency of the system parameter determination and imaging data post-processing.

In some embodiments, the scanning protocol may include the parameter value (also referred to as a fourth parameter value) of the system parameter corresponding to the target subject. The fourth parameter value may indicate whether the imaging data relating to the target subject needs to be post-processed. For example, the fourth parameter value may include a parameter value indicating that the imaging data relating to the target subject needs to be post-processed, and a parameter value indicating that the imaging data relating to the target subject does not need to be post-processed.

In some embodiments, the fourth parameter value may be represented as numbers, letters, symbols, etc. For example, the fourth parameter value of 1 may indicate that the imaging data (e.g., the target imaging data) relating to the target subject needs to be post-processed, and the fourth parameter value of 0 may indicate that the imaging data (e.g., the target imaging data) relating to the target subject does not need to be post-processed.

In some embodiments, the fourth parameter value of the system parameter may be determined based on a system default setting or set manually by the user. For example, the fourth parameter value of the system parameter may be pre-determined, and stored in a storage device (e.g., the storage device 150). The processing device 140 may retrieve the fourth parameter value of the system parameter corresponding to the target subject when the scanning protocol (or the system parameter) is determined.

In some embodiments, the processing device 140 may determine the fourth parameter value of the system parameter firstly, and then determine other parameter values (e.g., the first parameter value, the second parameter value, etc.). For example, if the fourth parameter value of the system parameter is determined as the value “1” indicating that the imaging data (e.g., the target imaging data) relating to the target subject needs to be post-processed, the processing device 140 may determine the other parameter values based on actual conditions. If the fourth parameter value of the system parameter is determined as the value “0” indicating that the imaging data (e.g., the target imaging data) relating to the target subject does not need to be post-processed, the processing device 140 may determine the first parameter value as the value “0” indicating the post-processing operation does not need to be performed on the target imaging data.

The scanning parameter may relate to one or more parameters that are used to direct an imaging device (e.g., the imaging device 110) to perform the medical scan on the target subject. Exemplary scanning parameters may include an FOV, a scanning region, a scanning sequence, parameter(s) of the imaging device, parameter(s) of the target subject, parameter(s) of the target imaging data, or the like, or any combination thereof.

In some embodiments, the scanning sequence may include a spin echo sequence, a fast spin echo sequence, an inversion recovery sequence, a gradient echo sequence, an echo planar imaging sequence, or the like, or any combination thereof.

In some embodiments, the parameter(s) of the imaging device may include hardware parameter(s), software parameter(s), etc., of the imaging device. A hardware parameter may refer to a parameter relating to hardware of the imaging device. For example, if the imaging device is a CT device, exemplary hardware parameters may include parameters relating to an X-ray generator, a filter, a collimator, a detector, an analog-to-digital converter, etc., of the CT device. A software parameter may refer to a parameter relating to software of the imaging device. Exemplary hardware parameters may include parameters relating to a version, an algorithm for processing the target imaging data, ctc.

In some embodiments, the parameter(s) of the target subject may include a gender, an age, a body shape, etc., of the target subject.

In some embodiments, the scanning parameter(s) may be determined based on a system default setting or set manually by the user. For example, parameter value(s) of the scanning parameter(s) may be pre-determined, and stored in a storage device (e.g., the storage device 150). The processing device 140 may retrieve the parameter value(s) of the scanning parameter(s) when the scanning protocol (or the system parameter) is determined.

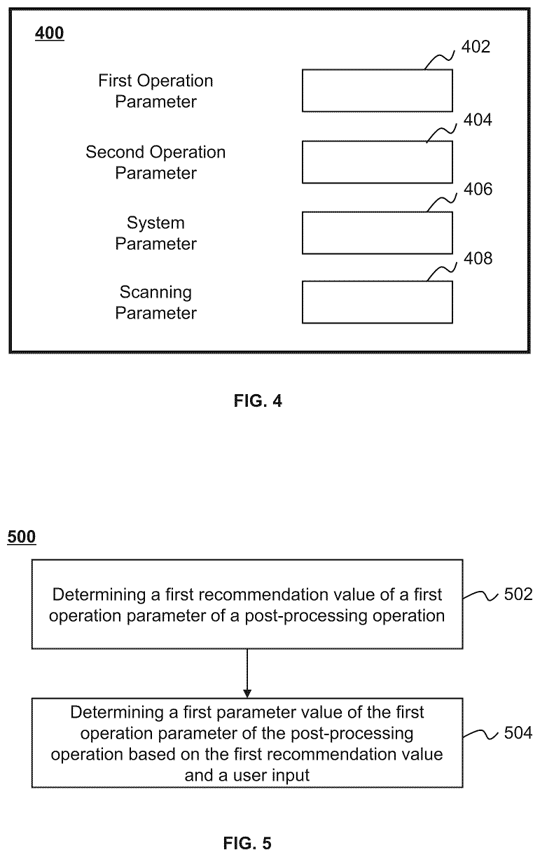

In some embodiments, the processing device 140 may display the scanning protocol of the medical scan of the target subject on a user interface for the user to read and/or adjust the scanning protocol. For example, referring to FIG. 4, FIG. 4 is a schematic diagram illustrating an exemplary user interface 400 according to some embodiments of the present disclosure. As illustrated in FIG. 4, the user interface 400 may include a first operation parameter of each of one or more post-processing operations, a second operation parameter of each of the one or more post-processing operations, a system parameter corresponding to a target subject, a scanning parameter, or the like, or any combination thereof. A block 402 may be used to display a first parameter value of the first operation parameter of each of the one or more post-processing operations. A block 404 may be used to display a third parameter value of the second operation parameter of each of the one or more post-processing operations. A block 406 may be used to display a fourth parameter value of the system parameter corresponding to the target subject. A block 408 may be used to display a parameter value of the scanning parameter. In some embodiments, after the first parameter value, the third parameter value, the fourth parameter value, and/or the parameter value of the scanning parameter are displayed, a user may check and/or adjust the first parameter value, the third parameter value, the fourth parameter value, and/or the parameter value of the scanning parameter through an input device. It should be noted that the description of the user interface 400 is merely for illustration, and is not intended to limit the scope of the present disclosure. For example, the user interface 400 may include other information of the scanning protocol of the medical scan of the target subject. As another example, the parameters and parameter values thereof may be arranged in any other manner on the user interface 400.

In 304, the processing device 140 (e.g., the obtaining module 220) may obtain the target imaging data of the target subject collected in the medical scan that is performed according to the scanning protocol.

The target imaging data may refer to imaging data that needs to be analyzed and/or post-processed.

In some embodiments, the processing device 140 may obtain the target imaging data from the imaging device (e.g., the imaging device 110) or a storage device (e.g., the storage device 150, a database, or an external storage) that stores the target imaging data of the target subject. For example, an imaging device (e.g., the imaging device 110) may perform the medical scan on the target subject according to the scanning protocol to collect the target imaging data of the target subject, and the processing device 140 may obtain the target imaging data from the imaging device. As another example, the processing device 140 may obtain the target imaging data from a third party. Therefore, an offline image processing can be realized.

In 306, the processing device 140 (e.g., the post-processing module 230) may perform at least part of the one or more post-processing operations on the target imaging data based on the scanning protocol.

In some embodiments, the at least part of the one or more post-processing operations may refer to post-processing operation(s) that need to be performed on the target imaging data specified in the scanning protocol. For example, after the target imaging data is obtained, the processing device 140 may determine the at least part of the one or more post-processing operations based on the scanning protocol (e.g., the first parameter value of the first operation parameter of each of the one or more post-processing operations, the fourth parameter value of the system parameter, etc.), and perform the at least part of the one or more post-processing operations on the target imaging data.

In some embodiments, the processing device 140 may perform the at least part of the one or more post-processing operations on the target imaging data based on the first parameter value of the first operation parameter of each of the one or more post-processing operations. For example, if the first parameter value of the first operation parameter of a post-processing operation indicates that the post-processing operation needs to be performed on the target imaging data (e.g., when the first parameter value is 1), the processing device 140 may perform the post-processing operation on the target imaging data. As another example, if the first parameter value of the first operation parameter of the post-processing operation indicates that the post-processing operation does not need to be performed on the target imaging data (e.g., when the first parameter value is 0), the processing device 140 may not perform the post-processing operation on the target imaging data.

Merely by way of example, when the post-processing operation is the organizational analysis, and the corresponding first parameter value is 1, the processing device 140 may perform the organizational analysis on the target imaging data. For instance, the processing device 140 may obtain a reconstruction image of the target subject by reconstructing the target imaging data, and perform the organizational analysis on the reconstruction image using an organizational analysis module. The organizational analysis module (e.g., a portion of parameters in the organizational analysis module) may be pre-determined and stored in the imaging device or the storage device, and the processing device 140 may retrieve the organizational analysis module from the imaging device or the storage device. When the first parameter value corresponding to the organizational analysis is 0, the processing device 140 may not perform the organizational analysis on the target imaging data.

As another example, when the post-processing operation is the ROI segmentation, and the corresponding first parameter value is 1, the processing device 140 may perform the ROI segmentation on the target imaging data to obtain one or more segmentation images. For instance, the processing device 140 may perform the ROI segmentation on the target imaging data (e.g., a reconstruction image) according to a configuration instruction. The configuration instruction may include any image processing algorithm and program instruction(s) thereof that can segment the ROI from the target imaging data. For example, the image processing algorithm may include an image segmentation algorithm, such as, a region-based segmentation algorithm, an edge-based segmentation algorithm, a wavelet transform segmentation algorithm, a mathematical morphology segmentation algorithm, a genetic algorithm-based segmentation algorithm, etc.

In some embodiments, the processing device 140 may perform the ROI segmentation on the target imaging data (e.g., the reconstruction image) according to a user instruction input by the user. For example, the target imaging data (e.g., the reconstruction image) may be displayed on a user terminal (e.g., a user interface of the terminal(s) 130), and a user (e.g., a doctor) may mark one or more ROIs (e.g., using a selection framework) on the target imaging data (e.g., the reconstruction image). A shape of the selection framework may include a rectangle, a circle, an ellipse, an irregular polygon, ctc. In some embodiments, the ROI segmentation may further include adjusting the ROI, such as, modifying the ROI (e.g., modifying a position, a shape, and/or an area of the ROI), adding the ROI, removing the ROI, etc.

Referring to FIG. 9, FIG. 9 is a schematic diagram illustrating an exemplary user interface 900 for ROI segmentation according to some embodiments of the present disclosure. As illustrated in FIG. 9, a reconstruction image of a target subject 950 may be displayed in the user interface 900, and the reconstruction image may include a first ROI 902, a second ROI 904, and a third ROI 906. In some embodiments, the first ROI 902, the second ROI 904, and the third ROI 906 may be further adjusted. For example, the second ROI 904 may be obtained by modifying a position, a shape, and an area of the first ROI 902. As another example, the third ROI 906 may be removed. By performing the ROI segmentation according to the configuration instruction and/or the user instruction, the flexibility, accuracy, and efficiency of the ROI segmentation may be improved. In addition, other post-processing operations may be performed on the one or more segmentation images, which improves the accuracy of the image analysis.

In some embodiments, the processing device 140 may perform one or more supplementary operations to perform the ROI segmentation. Exemplary supplementary operations may include zooming in, zooming out, flipping over, translating, etc., a portion of the target imaging data where the ROI is located, placing the ROI in a center of a field of view, or the like, or any combination thereof. Therefore, it is convenient for a user to observe whether to manually modify the ROI, which improves the accuracy and efficiency of the ROI segmentation.

As yet another example, when the post-processing operation is the noise reduction, and the corresponding first parameter value is 1, the processing device 140 may perform the noise reduction on the target imaging data. For instance, the processing device 140 may perform the noise reduction on the target imaging data using a denoising model, a filter kernel for denoising, etc. When the corresponding first parameter value of the noise reduction is 0, the processing device 140 may not perform the noise reduction on the target imaging data.

As still another example, when the post-processing operation is the resolution adjustment, and the corresponding first parameter value is 1, the processing device 140 may perform the resolution adjustment on the target imaging data. For instance, the processing device 140 may increase the image resolution or decrease the image resolution of the target imaging data. When the corresponding first parameter value is 0, the processing device 140 may not perform the resolution adjustment on the target imaging data.

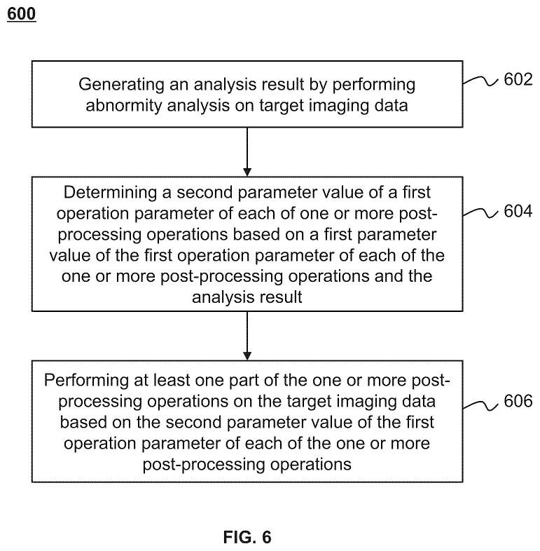

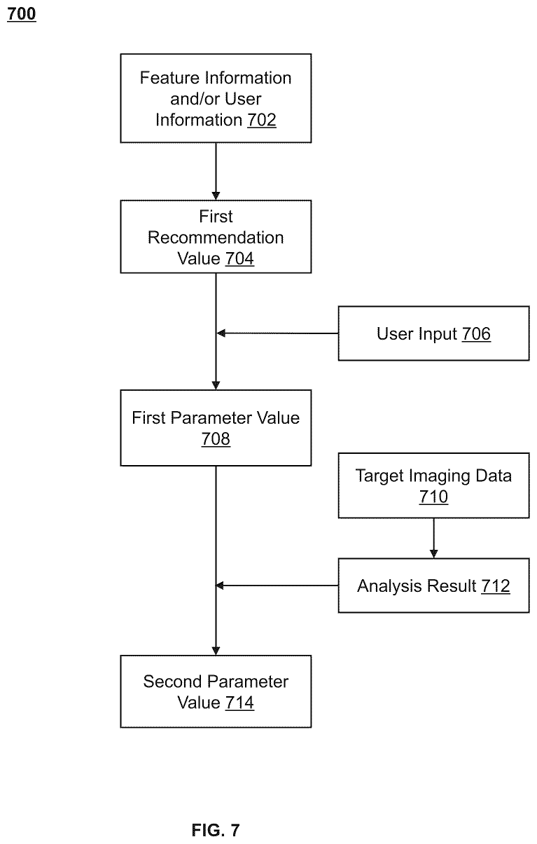

In some embodiments, after the target imaging data is obtained, the processing device 140 may determine a second parameter value of the first operation parameter of each of the one or more post-processing operations based on the first parameter value of the first operation parameter of each of the one or more post-processing operations and the target imaging data. The processing device 140 may further perform the at least one part of the one or more post-processing operations on the target imaging data based on the second parameter value of the first operation parameter of each of the one or more post-processing operations. For example, the processing device 140 may generate an analysis result by performing abnormity analysis on the target imaging data, and determine the second parameter value of the first operation parameter of each of the one or more post-processing operations based on the first parameter value of the first operation parameter of each of the one or more post-processing operations and the analysis result. The processing device 140 may perform the at least part of the one or more post-processing operations on the target imaging data based on the second parameter value of the first operation parameter of each of the one or more post-processing operations. More descriptions regarding the determination of the second parameter value of the first operation parameter may be found in elsewhere in the present disclosure (e.g., FIGS. 6 and 7, and the descriptions thereof).

In some embodiments, if it is determined that a specific post-processing operation needs to be performed, the processing device 140 may perform the post-processing operation on corresponding imaging data based on the third parameter value of the second operation parameter of the post-processing operation. For example, if the third parameter value of the second operation parameter of the post-processing operation indicates that the post-processing operation needs to be performed on the target imaging data, the processing device 140 may perform the post-processing operation on the target imaging data. As another example, if the third parameter value of the second operation parameter of the post-processing operation indicates that the post-processing operation needs to be performed on the post-processed imaging data generated by performing one or more other post-processing operations on the target imaging data, the processing device 140 may perform the post-processing operation on the post-processed imaging data.

Merely by way of example, if the third parameter value of the second operation parameter of the organizational analysis indicates that the organizational analysis needs to be performed on an ROI segmentation image, the processing device 140 may first perform the ROI segmentation on the target imaging data to generate the ROI segmentation image, and then perform organizational analysis on the ROI segmentation image.

In some embodiments, when the scanning protocol includes the system parameter corresponding to the target subject, the processing device 140 may determine whether the target imaging data needs to be post-processed based on the parameter value (i.e., the fourth parameter value) of the system parameter. If the target imaging data needs to be post-processed, the processing device 140 may perform the at least part of the one or more post-processing operations on the target imaging data based on the first parameter value of the first operation parameter of each of the one or more post-processing operations. If the target imaging data does not need to be post-processed, the processing device 140 may not perform any post-processing operation on the target imaging data.

In 308, the processing device 140 (e.g., the generation module 240) may generate an analysis report of the target subject based on post-processed imaging data.

The analysis report may include an organizational analysis result, an ROI segmentation result, detection information, or the like, or any combination thereof.

The organizational analysis result may include information regarding organizational content(s), information regarding organizational distribution(s), etc. For example, the organizational analysis result may include a fat analysis report, an analysis report of heart function, and other analysis reports used to provide detection information of the target subject. Merely by way of example, when the organizational analysis result includes the fat analysis report, the analysis report may include a fat content, a fat distribution, etc.

The ROI segmentation result may include information regarding the ROI. For example, the ROI segmentation result may include position information, contour information, shape information regarding the ROI and/or the one or more segmentation images of the ROI.

The detection information may include information, such as a size, a position, a severity degree, a shape, an ingredient, or the like, or any combination thereof, of the target subject (or the ROI of the target subject). Merely by way of example, the ROI may be a tumor, and the detection information of the ROI may include a size, a volume, a position, a severity degree, a stage, a type (e.g., benign or malignant), etc., of the tumor.

In some embodiments, the processing device 140 may automatically generate the analysis report of the target subject after the at least part of the one or more post-processing operations is performed on the target imaging data. For example, the processing device 140 may obtain a request for generating the analysis report from a user terminal (e.g., the terminal(s) 130), and generate the analysis report of the target subject based on the post-processed imaging data.

In some embodiments, the processing device 140 may generate the analysis report based on a report template. The report template may be preset based on a system default setting or set manually by a user. In some embodiments, the report template may include various items, such as, the organizational analysis result, the ROI segmentation result, the detection information, etc., of the target subject. The processing device 140 may fill the report template using the post-processed imaging data to generate the analysis report. For example, the post-processed imaging data may be ranked according to the report template. For instance, for 3D liver imaging data, image slices in the 3D liver imaging data may be ranked by a fat content from highest to lowest according to the report template.

In some embodiments, the processing device 140 may display the analysis report. For example, after the analysis report is obtained, the processing device 140 may display the analysis report through a display screen (e.g., a display screen of the terminal(s) 130). Exemplary display screens may include a liquid crystal display screen, an electronic ink display screen, etc.

In some embodiments, the user may further modify the analysis report through an input device. For example, the user may compare the target imaging data and one or more sets of post-processed imaging data generated by performing one or more post-processing operations on the target imaging data, so as to determine imaging data needs to be stored or determine imaging data needs to be saved in the analysis report.

In some embodiments, the processing device 140 may implement the process 300 in a multi-process manner. For example, the processing device 140 may process a plurality of sets of target imaging data simultaneously. As another example, the processing device 140 may simultaneously determine a scanning protocol of a medical scan of a first target subject, obtain target imaging data of a second target subject, perform at least part of the one or more post-processing operations on target imaging data of a third target subject, and generate an analysis report of a fourth target subject using multiple processors or threads. As still another example, all scanning protocols that need to be performed on a target subject may be added to a checklist, and the scanning protocols may be successively performed based on the checklist, regardless of whether imaging data corresponding to a previous scanning protocol has been reconstructed, whether the one or more post-processing operations have been performed on the imaging data corresponding to the previous scanning protocol, etc. By using the multi-process manner, the efficiency of the image processing may be improved.

In some embodiments, the processing device 140 may implement the process 300 on the target imaging data obtained from a third party (e.g., an imaging device other than the imaging device 110, a storage device other than the storage device 150, etc.). Merely by way of example, the processing device 140 may determine a scanning protocol of a target subject, obtain target imaging data from a third party, and perform at least part of the one or more post-processing operations on the target imaging data based on the scanning protocol. Therefore, the offline post-processing can be realized, thereby improving the convenience of the image processing.

According to some embodiments of the present disclosure, the scanning protocol of the medical scan of the target subject may be determined to obtain the target imaging data and/or process the target imaging data, which improves the reliability of the obtaining and processing of the target imaging data, and ensures the image quality of the target imaging data and/or the processed target imaging data, thereby improving the reliability of the post-processing operations and the image analysis. In addition, parameter values (e.g., the first parameter value and the third parameter value) relating to the post-processing operations may be determined automatically, which reduces the labor consumption and improves the efficiency of determining the scanning protocol and the image analysis. Alternatively, the parameter values may be determined according to the configuration instruction and/or the user instruction, which can semi-automatically perform the post-processing operations with user intervention, thereby improving the accuracy and efficiency of the image analysis. Moreover, the post-processing operations and the image analysis may be performed the target imaging data from any sources (e.g., the imaging device 110, the storage device 150, a third party, etc.), which can realize the online post-processing and/or the offline post-processing, thereby improving the convenience of the image analysis.

FIG. 5 is a flowchart illustrating an exemplary process 500 for determining a first parameter value of a first operation parameter according to some embodiments of the present disclosure. In some embodiments, the process 500 may be performed to achieve at least part of operation 302 as described in connection with FIG. 3.

In 502, the processing device 140 (e.g., the determination module 210) may determine a first recommendation value of a first operation parameter of a post-processing operation.

The first recommendation value may refer to a parameter value recommended for the first operation parameter of the post-processing operation. A type of the first recommendation value may be the same as the type of the first parameter value. For example, the first recommendation value may also indicate whether the post-processing operation needs to be performed on the target imaging data collected in a medical scan.

In some embodiments, the processing device 140 may determine the first recommendation value of the first operation parameter of the post-processing operation based on historical scanning protocols relating to the target subject. For example, if the first parameter value of the post-processing operation in one or more historical scanning protocols similar to the scanning protocol indicates that the post-processing operation needs to be performed on the target imaging data, the processing device 140 may determine that the first recommendation value is 1 (i.e., the post-processing operation is recommended to be performed on the target imaging data.

In some embodiments, the processing device 140 may determine the first recommendation value of the first operation parameter of the post-processing operation based on feature information relating to the target subject.

The feature information may refer to feature(s) of the target subject. Exemplary feature information may include physiological feature information, personal feature information, etc., of the target subject. For example, the physiological feature information may include an age, a height, a weight, a body temperature, a heart rate, a blood pressure, a blood oxygen saturation, a respiratory rate, a lung capacity, a blood sugar, an insulin level, etc., of the target subject. As another example, the personal feature information may include a medical history, a behavior history, a historical scanning condition, etc., of the target subject.

In some embodiments, the processing device 140 may determine the first recommendation value based on the feature information relating to the target subject. For example, when the feature information includes a heart rate exceeding a heart rate threshold, the processing device 140 may determine that the first recommendation value of a heart segmentation operation is 1 (i.e., the heart segmentation operation is recommended to be performed on the target imaging data). As another example, when the feature information includes a long-term smoking history, the processing device 140 may determine that the first recommendation value of the organizational analysis is 1 (i.e., the organizational analysis is recommended to be performed on the target imaging data or a portion of the target imaging data corresponding to the lung of the target subject).

In some embodiments, the processing device 140 may determine the first recommendation value based on user information. The user information may include a career, a department, a major, a field, preference information, etc., of a user (e.g., a doctor, a technician, etc.). For example, when the user information includes a cardiology department of the user, the processing device 140 may determine that the first recommendation value of the heart segmentation operation is 1 (i.e., the heart segmentation operation is recommended to be performed on the target imaging data).

In some embodiments, the first recommended value may also be determined in other manners. For example, the first recommended value may be determined based on the feature information relating to the target subject and/or the user information by using a machine learning model.

In 504, the processing device 140 (e.g., the determination module 210) may determine a first parameter value of the first operation parameter of the post-processing operation based on the first recommendation value and a user input.

The user input may include a confirmation or an adjustment of the first recommendation value. If the user input includes the confirmation of the first recommendation value, the first recommendation value may be designated as the first parameter value. If the user input includes the adjustment of the first recommendation value, the adjusted recommendation value may be designated as the first parameter value. Merely by way of example, the first recommendation value of the post-processing operation may be 1, the user may adjust the first recommendation value into 0, and the processing device 140 may determine that the first parameter value of the first operation parameter of the post-processing operation is 0.

According to some embodiments of the present disclosure, the first recommendation value may be determined automatically based on personalized information (e.g., the feature information relating to the target subject, the user information, etc.) automatically, which improves a matching degree between the first recommendation value and the target subject and/or the user. In addition, the first recommendation value may be provided to the user as reference, so that the first parameter value can be determined more efficiently.

FIG. 6 is a flowchart illustrating an exemplary process 600 for image processing according to some embodiments of the present disclosure. In some embodiments, the process 600 may be performed to achieve at least part of operation 306 as described in connection with FIG. 3.

In 602, the processing device 140 (e.g., the post-processing module 230) may generate an analysis result by performing abnormity analysis on target imaging data.

The abnormity analysis may include the analysis of whether a signal-to-noise ratio of the target imaging data is abnormal, whether the target imaging data includes image artifact, whether a status (e.g., a liver function) of a target subject is abnormal, or the like, or any combination thereof.

The analysis result may include whether the target imaging data of a target subject is abnormal, an abnormal condition of the target imaging data, an abnormal status of the target subject, or the like, or any combination thereof. For example, the analysis result may include that the signal-to-noise ratio of the target imaging data is abnormal, the target imaging data includes image artifact, the liver function of the target subject is abnormal, etc.

In some embodiments, the processing device 140 may generate the analysis result using an abnormity analysis model. For example, the processing device 140 may obtain the abnormity analysis model, and generate the analysis result by inputting the target imaging data into the abnormity analysis model.

In some embodiments, the abnormity analysis model may refer to a process or an algorithm used for generating the analysis result based on the target imaging data. The abnormity analysis model may be a trained machine learning model. Exemplary machine learning models may include a convolutional neural network (CNN) model, a recurrent neural network (RNN) model, a deep residual network (DRN) model, a long short term memory (LSTM) network model, a fully convolutional neural network (FCN) model, a generative adversarial network (GAN) model, a u-net model, a radial basis function (RBF) machine learning model, a DeepMask model, a SegNet model, a dilated convolution model, a conditional random fields as recurrent neural networks (CRFasRNN) model, a pyramid scene parsing network (pspnet) model, or the like, or any combination thereof.

In some embodiments, the processing device 140 may obtain the abnormity analysis model from a storage device (e.g., the storage device 150) of the imaging system 100 or a third-party database. In some embodiments, the abnormity analysis model may be generated by the processing device 140 or another computing device according to a machine learning algorithm. In some embodiments, the abnormity analysis model may be generated by training an initial model using a plurality of training samples. Each of the plurality of training samples may include sample imaging data of a sample subject and a sample analysis result of the sample imaging data. The sample imaging data of a training sample may be obtained using an imaging device (e.g., the imaging device 110). The sample analysis result may be determined manually by a user and used as a training label.

In some embodiments, the abnormity analysis model may include a plurality of sub-models, and each of the plurality of sub-models may correspond to one type of abnormity condition. For example, the abnormity analysis model may include two sub-models. A first sub-model may be configured to determine the abnormal condition of the target imaging data, and a second sub-model may be configured to determine the abnormal status of the target subject. As another example, the abnormity analysis model may include a plurality of sub-models, and the plurality of sub-models may be configured to determine whether the signal-to-noise ratio of the target imaging data is abnormal, whether the target imaging data includes image artifact, whether the liver function of the target subject is abnormal, respectively.

In 604, the processing device 140 (e.g., the post-processing module 230) may determine a second parameter value of a first operation parameter of each of one or more post-processing operations based on a first parameter value of the first operation parameter of each of the one or more post-processing operations and the analysis result.

The second parameter value may be similar to the first parameter value and also indicate whether the post-processing operation needs to be performed on the target imaging data collected in the medical scan. The difference between the first parameter value and the second parameter value is that the first parameter value is determined based on feature information of the target subject or manually by the user, and the second parameter value is further determined by taking the abnormity condition of the target imaging data and/or the target subject into consideration. Therefore, the accuracy of the second parameter value may be higher than the accuracy of the first parameter value, which may improve the processing accuracy of the target imaging data.

In some embodiments, the processing device 140 may maintain or modify the first parameter value of a post-processing operation as the corresponding second parameter value based on the abnormity condition included in the analysis result. For example, if the analysis result indicates that the liver function of the target subject is abnormal, but the first parameter value of the organizational analysis is 0, the processing device 140 may modify the first parameter value as 1 (i.e., determine that the second parameter value of the organizational analysis is 1). As another example, if the analysis result indicates that the liver function of the target subject is normal, but the first parameter value of the organizational analysis is 1, the processing device 140 may modify the first parameter value as 1 (i.e., determine that the second parameter value the organizational analysis is 1).

In 606, the processing device 140 (e.g., the post-processing module 230) may perform at least part of the one or more post-processing operations on the target imaging data based on the second parameter value of the first operation parameter of each of the one or more post-processing operations.

For example, the processing device 140 may determine the at least part of the one or more post-processing operations that need to be performed based on the second parameter value of the first operation parameter of each of the one or more post-processing operations, and perform the at least part of the one or more post-processing operations on the target imaging data. The at least part of the one or more post-processing operations may be performed in a similar manner as how the at least part of the one or more post-processing operations are performed as described in FIG. 3.

According to some embodiments of the present disclosure, the analysis result may be generated by performing the abnormity analysis on the target imaging data, and the second parameter value may be determined based on the first parameter value and the analysis result, which optimizes the parameter value of the first operation parameter by taking the abnormity condition of the target imaging data and/or the target subject into consideration, thereby improving the accuracy of the post-processing operations on the target imaging data.

FIG. 7 is a schematic diagram illustrating an exemplary process 700 for determining a parameter value of a first operation parameter according to some embodiments of the present disclosure.

As illustrated in FIG. 7, feature information relating to a target subject and/or user information 702 may be obtained. A first recommendation value 704 of a first operation parameter of each of one or more post-processing operations may be determined based on the feature information relating to the target subject and/or the user information 702. A first parameter value 708 of the first operation parameter of each of the one or more post-processing operations may be determined based on the first recommendation value 704 and a user input 706.

In some embodiments, after target imaging data 710 of the target subject is obtained, an analysis result 712 may be generated by performing abnormity analysis on the target imaging data 710. A second parameter value 714 of the first operation parameter of each of the one or more post-processing operations may be determined by updating the first parameter value 708 based on the analysis result 712.

FIG. 8 is a flowchart illustrating an exemplary process 800 for determining a third parameter value of a second operation parameter according to some embodiments of the present disclosure. In some embodiments, the process 800 may be performed to achieve at least part of operation 302 as described in connection with FIG. 3.

In 802, the processing device 140 (e.g., the determination module 210) may determine a second recommendation value of a second operation parameter of a post-processing operation.

The second recommendation value may refer to a parameter value recommended for the second operation parameter of the post-processing operation. A type of the second recommendation value may be the same as the type of the third parameter value. For example, the second recommendation value may also indicate the type of data based on which the post-processing operation is performed.

In some embodiments, the processing device 140 may determine the second recommendation value of the post-processing operation based on historical scanning protocols set by a user. For example, if the third parameter value of the post-processing operation in one or more historical scanning protocols indicates that the post-processing operation is performed on the target imaging data, the processing device 140 may determine that the second recommendation value is 0 (i.e., the post-processing operation is recommended to be performed on the target imaging data). As another example, if the third parameter value of the post-processing operation in one or more historical scanning protocols indicates that the post-processing operation is performed on an ROI segmentation image, the processing device 140 may determine that the second recommendation value is 1 (i.e., the post-processing operation is recommended to be performed on segmented imaging data generated by performing the ROI segmentation on the target imaging data).

In some embodiments, the second recommendation value of the second operation parameter of the post-processing operation may be determined in a similar manner as how the first recommendation value is determined as described in FIG. 5. For example, the second recommendation value may be determined based on feature information relating to the target subject. As another example, the second recommendation value may be determined based on user information.

In 804, the processing device 140 (e.g., the determination module 210) may determine a third parameter value of the post-processing operation based on the second recommendation value and a user input.

The user input may include a confirmation or an adjustment of the second recommendation value. If the user input includes the confirmation of the second recommendation value, the second recommendation value may be designated as the third parameter value. If the user input includes the adjustment of the second recommendation value, the adjusted recommendation value may be designated as the third parameter value. The third parameter value may be determined in a similar manner as how the first parameter value is determined as described in FIG. 5.

Processes 300, 500, 600, and 800 may be implemented in the imaging system 100 illustrated in FIG. 1. For example, the processes 300, 500, 600, and 800 may be stored in the storage device 150 in the form of instructions (e.g., an application), and invoked and/or executed by the processing device 140. The operations of the illustrated process presented below are intended to be illustrative. In some embodiments, the processes 300, 500, 600, and 800 may be accomplished with one or more additional operations not described, and/or without one or more of the operations discussed. Additionally, the order in which the operations of the processes 300, 500, 600, and 800 as illustrated in FIGS. 3, 5, 6, and 8 as described below is not intended to be limiting.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended for those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this disclosure are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed subject matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A method for image processing, implemented on a computing device having at least one processor and at least one storage device, the method comprising:
determining a scanning protocol of a medical scan of a target subject, the scanning protocol including a first parameter value of a first operation parameter of each of one or more post-processing operations, the first operation parameter of a post-processing operation relating to whether the post-processing operation needs to be performed on target imaging data collected in the medical scan;
obtaining the target imaging data of the target subject collected in the medical scan that is performed according to the scanning protocol; and
performing at least part of the one or more post-processing operations on the target imaging data based on the first parameter value of the first operation parameter of each of the one or more post-processing operations.

2. The method of claim 1, wherein the one or more post-processing operations include at least one of an organizational analysis, a region of interest (ROI) segmentation, a noise reduction, or a resolution adjustment.

3. The method of claim 1, wherein the scanning protocol further includes a parameter value of a system parameter corresponding to the target subject, the system parameter relating to whether imaging data relating to the target subject needs to be post-processed, and the performing at least part of the one or more post-processing operations on the target imaging data includes:
determining whether the target imaging data needs to be post-processed based on the parameter value of the system parameter;
in response to determining that the target imaging data needs to be post-processed, performing the at least part of the one or more post-processing operations on the target imaging data based on the first parameter value of the first operation parameter of each of the one or more post-processing operations.

4. The method of claim 1, wherein the first parameter value of the first operation parameter of each of the one or more post-processing operations is determined by:
for each of the one or more post-processing operations,
determining a first recommendation value of the first operation parameter of the post-processing operation; and
determining the first parameter value of the first operation parameter of the post-processing operation based on the first recommendation value and a user input.

5. The method of claim 4, wherein the determining a first recommendation value of the first operation parameter of the post-processing operation includes:
determining the first recommendation value of the first operation parameter of the post-processing operation based on feature information relating to the target subject.

6. The method of claim 1, wherein the performing at least part of the one or more post-processing operations on the target imaging data based on the first parameter value of the first operation parameter of each of the one or more post-processing operations includes:
generating an analysis result by performing abnormity analysis on the target imaging data;
determining a second parameter value of the first operation parameter of each of the one or more post-processing operations based on the first parameter value of the first operation parameter of each of the one or more post-processing operations and the analysis result; and
performing the at least part of the one or more post-processing operations on the target imaging data based on the second parameter value of the first operation parameter of each of the one or more post-processing operations.

7. The method of claim 6, wherein the generating an analysis result by performing an abnormity analysis on the target imaging data includes:
obtaining an abnormity analysis model, the abnormity analysis model being a trained machine learning model; and
generating the analysis result based on the target imaging data using the abnormity analysis model.

8. The method of claim 1, wherein the scanning protocol includes a third parameter value of a second operation parameter of each of the one or more post-processing operations, the second operation parameter of a post-processing operation relating to the type of data based on which the post-processing operation is performed.

9. The method of claim 8, wherein the third parameter value of the second operation parameter of each of the one or more post-processing operations is determined by:
for each of the one or more post-processing operations,
determining a second recommendation value of the second operation parameter of the post-processing operation; and
determining the third parameter value of the post-processing operation based on the second recommendation value and a user input.

10. The method of claim 1, further comprising:
generating an analysis report of the target subject based on post-processed imaging data.

11. The method of claim 1, further comprising:
displaying the scanning protocol of the medical scan via a user interface.

12. A system for image processing, comprising:
at least one storage device including a set of instructions; and
at least one processor configured to communicate with the at least one storage device, wherein when executing the set of instructions, the at least one processor is configured to direct the system to perform operations including:
determining a scanning protocol of a medical scan of a target subject, the scanning protocol including a first parameter value of a first operation parameter of each of one or more post-processing operations, the first operation parameter of a post-processing operation relating to whether the post-processing operation needs to be performed on target imaging data collected in the medical scan;
obtaining the target imaging data of the target subject collected in the medical scan that is performed according to the scanning protocol; and
performing at least part of the one or more post-processing operations on the target imaging data based on the first parameter value of the first operation parameter of each of the one or more post-processing operations.

13. The system of claim 12, wherein the one or more post-processing operations include at least one of an organizational analysis, a region of interest (ROI) segmentation, a noise reduction, or a resolution adjustment.

14. The system of claim 12, wherein the scanning protocol further includes a parameter value of a system parameter corresponding to the target subject, the system parameter relating to whether imaging data relating to the target subject needs to be post-processed, and the performing at least part of the one or more post-processing operations on the target imaging data includes:
determining whether the target imaging data needs to be post-processed based on the parameter value of the system parameter;
in response to determining that the target imaging data needs to be post-processed, performing the at least part of the one or more post-processing operations on the target imaging data based on the first parameter value of the first operation parameter of each of the one or more post-processing operations.

15. The system of claim 12,
wherein the first parameter value of the first operation parameter of each of the one or more post-processing operations is determined by:
for each of the one or more post-processing operations,
determining a first recommendation value of the first operation parameter of the post-processing operation; and
determining the first parameter value of the first operation parameter of the post-processing operation based on the first recommendation value and a user input.

16. The system of claim 15, wherein the determining a first recommendation value of the first operation parameter of the post-processing operation includes:
determining the first recommendation value of the first operation parameter of the post-processing operation based on feature information relating to the target subject.

17. The system of claim 12, wherein the performing at least part of the one or more post-processing operations on the target imaging data based on the first parameter value of the first operation parameter of each of the one or more post-processing operations includes:
generating an analysis result by performing abnormity analysis on the target imaging data;
determining a second parameter value of the first operation parameter of each of the one or more post-processing operations based on the first parameter value of the first operation parameter of each of the one or more post-processing operations and the analysis result; and
performing the at least part of the one or more post-processing operations on the target imaging data based on the second parameter value of the first operation parameter of each of the one or more post-processing operations.

18. The system of claim 17, wherein the generating an analysis result by performing an abnormity analysis on the target imaging data includes:
obtaining an abnormity analysis model, the abnormity analysis model being a trained machine learning model; and
generating the analysis result based on the target imaging data using the abnormity analysis model.

19. The system of claim 12, wherein the scanning protocol includes a third parameter value of a second operation parameter of each of the one or more post-processing operations, the second operation parameter of a post-processing operation relating to the type of data based on which the post-processing operation is performed.

20-22. (canceled)

23. A non-transitory computer readable medium, comprising executable instructions that, when executed by at least one processor, direct the at least one processor to perform a method, the method comprising:
determining a scanning protocol of a medical scan of a target subject, the scanning protocol including a first parameter value of a first operation parameter of each of one or more post-processing operations, the first operation parameter of a post-processing operation relating to whether the post-processing operation needs to be performed on target imaging data collected in the medical scan;
obtaining the target imaging data of the target subject collected in the medical scan that is performed according to the scanning protocol; and
performing at least part of the one or more post-processing operations on the target imaging data based on the first parameter value of the first operation parameter of each of the one or more post-processing operations.

24. (canceled)
